(19) United States
(12) Patent Application Publication
(10) Pub. No.: US 2010/0130959 A1
(43) Pub. Date: May 27, 2010

(54) DEVICE AND METHOD FOR DELIVERY OF THERAPEUTIC AGENTS VIA ARTIFICIAL INTERNAL IMPLANTS

(75) Inventors: Lawrence M. Boyd, Durham, NC (US); Samuel B. Adams, JR., Durham, NC (US); Matthew R. Penny, Clayton, NC (US)

Correspondence Address: MOORE & VAN ALLEN PLLC, P.O. BOX 13706, Research Triangle Park, NC, 27709, US

(73) Assignee: PALMETTO BIOMEDICAL, INC., Durham, NC (US)

(21) Appl. No.: 12/580,190

(22) Filed: Oct. 15, 2009

Related U.S. Application Data
(60) Provisional application No. 61/105,659, filed on Oct. 15, 2008.

(51) Int. Cl. A61M 31/00

(52) U.S. Cl. 604/513; 604/93.01; 604/246; 604/272; 604/523; 604/500

(57) ABSTRACT

A device is provided for use with a medical implant for delivering an agent to a designated site of action in a body of a patient. The agent-delivery device comprises a body member adapted to be secured to the medical implant and an agent-delivery component associated with the body member. The agent-delivery component includes a therapeutic agent for treating the body of the patient. The agent-delivery component is configured to release the therapeutic agent after implantation in the body of the patient.

### CROSS-REFERENCES

This application is related to U.S. provisional application No. 61/105,659, filed Oct. 15, 2008, entitled “Delivery of Therapeutic Agents Via Artificial Internal Implants”, naming Lawrence M. Boyd, Samuel B. Adams, Jr., and Matthew R. Penny as inventors. The contents of the provisional application are incorporated herein by reference in their entirety, and the benefit of the filing date of the provisional application is hereby claimed for all purposes that are legally served by such claim for the benefit of the filing date.

### BACKGROUND

This invention relates generally to the delivery of therapeutic agents via artificial biomedical implants, and more particularly to an agent-delivery device adaptable to an internal biomedical implant.

There are many applications in which it is desirable to locally deliver a therapeutic agent adjacent to a biomedical implant such as a fracture plate, spinal rod or total joint prosthesis. For example, for growth factor delivery to secure accelerated bony fusion in a spinal fusion or fracture repair application, local delivery is necessary to concentrate the inductive agent at the site at which bone healing is desired. Another area in which local delivery would be advantageous involves the local delivery of an agent capable of reducing local pain and inflammation (e.g., an analgesic agent, therapeutic protein or antibody) alone or in concert with a surgical procedure such as a bony fusion. Finally, one area of great need involves local delivery of antibiotics for the treatment of implant associated infections.

Infections associated with surgical implants are generally difficult to manage because they require long periods of antibiotic therapy and repeated surgical procedures. Infections related to orthopedic devices and ventricular shunts often result in serious disabilities. Infected joint prosthesis occur between in more than ten thousand clinical cases per year in the United States, while infected fracture fixation devices (e.g., fracture plates and intramedullary rods) are even more widespread, there were nearly 100,000 infected fracture fixation implants in the United States in 2004 (Darouche, 2004). On average, about 5% of initially inserted internal fixation devices become infected. The infection rate for open fractures (those that involve compromise of the skin barrier) may exceed 30%. The cost to treat these infected implant sites is a significant cost to the healthcare system. For example, costs to treat spinal implant infection range from $40,000 to $400,000, depending on the severity and duration of the infection.

One significant challenge associated with the treatment of implant associated infections is the formation of a bacterial biofilm on the surface of the prosthesis. Bacteria biofilms involve the clustering of the microorganisms together in a highly hydrated extracellular matrix called a glycocalyx. Implants may be colonized acutely by perioperative airborne, skin- or surgeon-related bacteria seeded during surgery, or may adhere to the prosthesis via blood borne (hematogenous) pathogens at a later time. After attachment on the biomaterial surface, bacteria multiply and physiologically transform into a “biofilm” community. These biofilms are difficult to treat with systemic antibiotics for multiple reasons, including the quiescent nature of the bacteria in the biofilm community, poor vascularity of the biofilm, and its resistance to drug diffusion into the protein matrix (glycocalyx) formed by bacteria on the implant surface. Depletion of metabolic substances or waste product accumulation in biofilms also causes the microbes to enter into a slow growing or stationary phase, rendering them up to 1,000 times more resistant to most antimicrobial agents.

The nature of the surgical intervention to treat the infected device depends on the type of device, the presence or absence of bony union (for fracture fixation and spinal instrumentation devices) and the patient's underlying condition. For stable implants, debridement of the implant site, copious irrigation, high dose parenteral antibiotics and retention of the device with long-term (sometimes lifetime) oral antibiotic treatment is common. Surgical removal of the implant may be necessary to remove the source of the infection in the absence of a means of locally delivering high doses of therapeutic antibiotics, even in cases where the implant is still required for structural or functional performance. An additional follow-up procedure may be required to place a second implant once the infection is adequately treated.

Implant associated infections are often acquired in the hospital or surgical center. Federal (Medicare and Medicaid) and private insurers expend upwards of $1 billion treating hospital acquired, implant associated infections. This provides strong incentive and motivation for developing systems and methods for treating active infections and for preventing infection around medical devices.

A variety of methods are currently utilized to treat implant associated infection. These include the use of systemic prophylactic (pre- and post-operative) and post-infection antibiotics, delivery of antibiotic loaded PMMA bone cement, delivery of antibiotic loaded biomaterials, and active and passive surface coatings of the medical device prior to insertion. The most common method is to use systemic antibiotic therapy. However, these have been found to be expensive, prone to complications and very often not successful. One concern in delivering an antibiotic via the systemic route (oral, parenteral) involves the generally poor vascularity of the implant site, such as a bone fracture in the case of internal fixation implants. In order to deliver local therapeutic doses, it may be necessary to deliver high, and potentially toxic, levels of the antibiotic. The literature strongly supports the effectiveness of local treatment compared to systemic routes. This has been a major driving force toward developing methods to locally deliver a therapeutic agent. The local concentrations of antibiotic that can be achieved with local application cannot be achieved with systemic delivery, due to the toxic side effects that most antibiotics produce at such high systemic concentrations.

Another common method for treating implant associated infection, especially for joint replacement arthroplasty and large bony defects, has been the use of antibiotic impregnated bone cement (e.g., polymethylmethacrylate, PMMA). The antibiotic loaded cement may be mixed at the time of surgery, or a specially sized PMMA spacer may be used following removal of the prosthetic hip or knee replacement. In bone defects, for example with osteomyelitis, bone cement beads may be packed into the defect to increase surface-to-volume ratio for antibiotic delivery. For joint replacements, a two-stage replacement approach may be used, where the infected implant is removed and replaced by a biomaterial spacer until the infection is treated and a second prosthesis can be placed.

There are multiple concerns associated with the use of antibiotic-containing bone cement. Antibiotics may be slowly released over the first 4 weeks, after which a sub-therapeutic dose of the antibiotic may be locally present. There are concerns that the lower dose of antibiotic in later time points, below the minimal inhibitory concentration (MIC) of resident bacteria, may lead to the formation of antibiotic-resistant strains of bacteria around the implant. Also, the bone cement is a two part system that may have residual toxic components, which also undergoes a highly exothermic reaction, both aspects capable of killing local bone cells needed for healing.

Other biomaterials have also been proposed for local delivery of antibiotics. These carriers include collagen scaffolds, bone substitutes (calcium based biomaterials) and allograft bone with incorporated antibiotic agents. For fracture treatment, placing these biomaterials in addition to the extensive hardware used to treat the fracture, and the need to maintain the material adjacent the implant site, have limited their utility in trauma and spine applications.

Implant coatings have been proposed as a means of reducing bacterial biofilm formation. Providing metal implants commonly used for internal fixation or spine surgery with a coating that contains and releases an antibacterial or antiseptic substance after surgery has been an appealing solution to the problem of implant associated infection. Antiseptic coatings such as silver ions and chlorhexidine/chloroxylenol may be immobilized on the implant surface. The main rationale for the use of an antiseptic instead of an antibiotic is the lower potential for developing resistant bacterial strains. Other efforts have involved the coating of the implant with a resorbable polymer coating or film loaded with an antibiotic or antiseptic agent. Animal studies have demonstrated the potential utility of the use of a resorbable biomaterial for local delivery. For example, Kalicke and coauthors reported in 2006 that the use of an antibacterial (Rifampicin and fusidic acid) and biodegradable (poly-1-lactide) coating on titanium fracture fixation plates resulted in a significant reduction in infection rate in an animal model (“Effect of infection resistance of a local antiseptic and antibiotic coating on osteosynthesis implants: an in vitro and in vivo study” Journal of Orthopaedic Research August 2006, pp. 1622-1640). Pilot clinical studies have been performed using polymer/antibiotic coated intramedullary nails for enhanced fracture repair (Schmidmaier, et al., 2006).

Others have proposed to modify the implant by adding channels or openings in the implant that can be filled with a drug-eluting biomaterial. The concept of machining channels into the implant for receipt of a drug eluting biomaterial or gel has been proposed. Concerns with these methods involve the need to prospectively modify the implants, the potential effect of these material modifications on the strength of the device and the potential for pockets or channels to harbor microbes.

For the foregoing reasons, there is a need for local and sustained delivery of therapeutic agents within the body of a patient. The new device should be easily adaptable to medical implants, such as bone fixation implants, spinal fixation implants or reconstructive prostheses.

### SUMMARY

A device is provided for use with a medical implant for delivering an agent to a designated site of action in a body of a patient. The agent-delivery device comprises a body member adapted to be secured to the medical implant and an agent-delivery component associated with the body member. The agent-delivery component includes a therapeutic agent for treating the body of the patient. The agent-delivery component is configured to release the therapeutic agent after implantation in the body of the patient.

A system is also provided for use as a medical implant. The medical implant system comprises a medical implant and a device for delivering an agent to a designated site of action in a body of a patient. The agent-delivery device comprises a body member configured to be secured to the medical implant and an agent-delivery component associated with the body member. The agent-delivery component includes a therapeutic agent for treating the body of the patient. The agent-delivery component is configured to release the therapeutic agent after implantation in the body of the patient.

Further, a method is provided for delivering a therapeutic agent to a site within the body of a patient adjacent to a medical implant. The method comprising the steps of providing a medical implant, a therapeutic agent to be delivered to a site of action within the body of a patient, and an agent-delivery device. The therapeutic agent is operatively associated with the agent-delivery device such that the therapeutic agent is arranged to delivery a therapeutically effective amount of the agent to the site. Further steps include securing the agent-delivery device to the medical implant and surgically implanting the medical implant.

### BRIEF DESCRIPTION OF THE FIGURES

For a more complete understanding of the present invention, reference should now be had to the embodiments shown in the accompanying drawings and described below. In the drawings:

FIG. 1 is an elevation view of an embodiment of an agent-delivery device adapted to an internal fracture fixation plate secured to a bone fracture.

FIG. 2A is a top perspective view of an embodiment of an agent-delivery device.

FIG. 2B is a top plan view of the agent-delivery device shown in FIG. 2A.

FIG. 2C is a side elevation view of the agent-delivery device shown in FIG. 2A.

FIG. 2D is a bottom plan view of the agent-delivery device shown in FIG. 2A.

FIG. 2E is an end view of the agent-delivery device shown in FIG. 2A.

FIG. 3 is a cross-section view of the agent-delivery device shown in FIG. 2A adapted to an internal fracture fixation plate.

FIG. 4 is an exploded perspective view in partial cross-section of another embodiment agent-delivery device for use with an internal fracture fixation plate secured to a bone fracture.

FIG. 5 is a perspective view in partial cross-section of the agent-delivery device as shown in FIG. 4.

FIG. 6 is a exploded perspective view of the agent-delivery device as shown in FIG. 4 and an internal fracture fixation plate and fasteners for securing to a bone fracture.

FIG. 7 is a bottom perspective view of another embodiment of an agent-delivery device.

FIG. 8 is a cross-section of a bottom perspective view of the agent-delivery device shown in FIG. 7 in place on a fracture fixation plate.

FIG. 9 is a perspective view of the agent-delivery device as shown in FIG. 8.

FIG. 10A is a top perspective view of a snap-in embodiment of an agent-delivery device.

FIG. 10B is a top plan view of the agent-delivery device shown in FIG. 10A.

FIG. 10C is a side elevation view of the agent-delivery device shown in FIG. 10A.

FIG. 10D is a bottom plan view of the agent-delivery device shown in FIG. 10A.

FIG. 10E is an end view of the agent-delivery device shown in FIG. 10A.

FIG. 11 is a longitudinal cross-section view of the agent-delivery device shown in FIGS. 10A-10E adapted to an internal fracture fixation plate secured to a bone fracture.

FIG. 12 is an elevation view of the agent-delivery device shown in FIG. 11.

FIG. 13 is an elevation view of a total hip joint arthroplasty implant including the agent-delivery device as shown in FIGS. 10a-10E.

FIG. 14 is perspective view of an embodiment of an agent-delivery device adapted to a fracture fixation plate.

FIG. 15 is a cross-section view of the agent-delivery device shown in FIGS. 10A-10E adapted to an internal fracture fixation plate and including a carrier.

FIG. 16 is a cross-section view of the agent-delivery device shown in FIG. 7 adapted to an internal fracture fixation plate and including a carrier.

FIG. 17 is an exploded perspective view of an embodiment of an agent-delivery device and a rod for a spinal fusion construct.

FIG. 18 is a cross-section view of the agent-delivery device shown in FIG. 17 adapted to the rod.

FIG. 19 is an elevation view of the agent-delivery device shown in FIG. 17 adapted to the rod of a spinal fusion construct in an installed position on vertebrae.

FIG. 20A is a top plan view of a two-part embodiment of an agent-delivery device.

FIG. 20B is a bottom plan view of the agent-delivery device shown in FIG. 20A.

FIG. 20C is a side elevation view of the agent-delivery device shown in FIG. 20A.

FIG. 20D is an end elevation view of the agent-delivery device shown in FIG. 20A.

FIG. 20E is another end elevation view of the agent-delivery device shown in FIG. 20A.

FIG. 20F is another side elevation view of the agent-delivery device shown in FIG. 20A.

FIG. 21 is an exploded perspective view of the of the agent-delivery device shown in FIGS. 20A-20F.

FIG. 22 is a top perspective view of the of the agent-delivery device shown in FIG. 21 when assembled.

FIG. 23 is a cross-section view of the agent-delivery device shown in FIG. 21 in place on a fracture fixation plate.

FIG. 24 is a top perspective view of the of the agent-delivery device shown in FIG. 21 in place on a fracture fixation plate secured to a bone fracture.

FIG. 25 is schematic top plan view of an embodiment of an agent-delivery device including a reservoir and valve and an external locator.

### DESCRIPTION

As used herein, the terms “therapeutic agent” or “agent” are used interchangeably and refer to a compound or composition of matter which, when presented to an organism, human or animal, induces a desired pharmacologic or physiologic effect by local or systemic action. For example, the therapeutic agent includes one or more compounds or composition of matter providing enhanced bone density or bone growth, anti-infection, anti-inflammation or pain relief to the area proximal to the implant.

As used herein, the term “therapeutically effective amount” refers to an amount of a therapeutic agent that is nontoxic but sufficient to provide a desired effect. For example, a therapeutically effective amount is an amount sufficient to measurably decrease the symptom or etiology of a bone tissue trauma or to measurably enhance the rate of the targeted cell division, cell migration or cell attachment as necessary to accelerate bone healing and quality of the bone formed in response to injury. The therapeutically effective amount varies according to the patient's presentation, sex, age and weight, the rate of administration, the nature of the condition and any treatments which may be associated therewith, or any concurrent related or unrelated treatments or conditions of the patient. Therapeutically effective amounts can be determined without undue experimentation by any person skilled in the art or by following the exemplary guidelines set forth herein.

As used herein, the term “absorbable” or variations thereof mean the ability of a tissue-compatible material to degrade or biodegrade at some time after implantation into products that are eliminated from the body or metabolized therein. Thus, as used herein, “absorbability” means that the material is capable of being absorbed, either fully or partially, by tissue by cellular or biochemical means when implanted into a human or animal. The absorption time may vary depending on the particular uses and tissues involved.

As used herein, the term “non-absorbable” or variations thereof mean completely or substantially incapable of being absorbed, either fully or partially, by tissue after introduction to the subject.

Certain terminology is used herein for convenience only and is not to be taken as a limitation on the invention. For example, words such as “upper,” “lower,” “left,” “right,” “horizontal,” “vertical,” “upward,” and “downward” merely describe the configuration shown in the FIGs. Indeed, the components may be oriented in any direction and the terminology, therefore, should be understood as encompassing such variations unless specified otherwise.

Described herein are devices and methods for the local delivery of therapeutic agents to a site of bone fracture, healing or fixation and to surrounding tissues, allowing for immediate, continuous or sustained delivery of therapeutic agents, such as those used to prevent infection or to enhance the tissue healing process. In one aspect, an agent-delivery device is provided that securely adapts to an internal medical implant. Adaptation of the agent-delivery device to the medical implant may be implemented prior to or following implantation of the medical implant or other medical device, and may involve secure or reversible fixation.

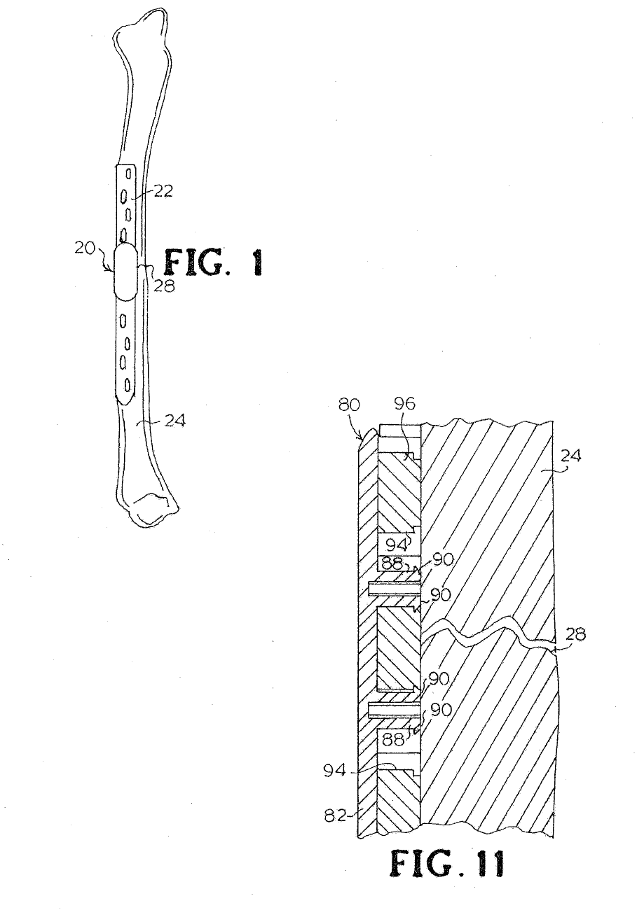

Referring now to the drawings, wherein like reference numerals designate corresponding or similar elements throughout the several views, an agent-delivery device for delivering therapeutic agents for use with an internal fracture fixation plate is shown in FIG. 1, and generally designated at 20. As is conventional, the plate 22 is fastened to a bone 24 using screws 26 such that the plate 22 spans either side of a fracture 28 or repair site. The agent-delivery device 20 is securely adaptable to the plate, allowing for a highly localized delivery of one or more therapeutic agents in the area around the plate 22. The term “securely adaptable” includes any fastening or securing means. Thus, it is understood that multiple means of adaptation are anticipated that can be used to attach the agent-delivery device to a wide range of medical implants, and is inclusive of securement means providing relative motion of the agent-delivery device relative to the medical implant, such as sliding along an axis of the plate 22 shown in FIG. 1.

The agent-delivery device can be formed of either synthetic or natural materials, including, but not limited to, thermoplastics, thermoset polymers, elastomers, rubbers, or woven or non-woven composite materials. The agent-delivery device may be, for example, any suitable molded form of a polymeric, plastic foam (including open celled foam), woven composite or non-woven composite, mixtures thereof, or the like. In particular, a suitable agent-delivery device may thus be prepared, for example, from Nylon, a polyolefin, such as polyethylene, including UHMW polyethylene, structural plastics such as PEEK (polyetheretherketone), polysulfone, polypropylene, ethylene propylene copolymers, and ethylene butylene copolymers, polyurethanes, polyurethane foams, polystyrenes, plasticized polyvinylchlorides, polyesters, Delrin polyacetal, and polyamides, and homopolymer and copolymers of the above. It is understood that the agent-delivery device may assume a variety of shapes as necessary to accommodate and adapt to a variety of fixation plates.

The agent-delivery device may be absorbable or non-absorbable. In one aspect, the agent-delivery device may be formed from an absorbable polymer, such as a polymer, copolymer, or homopolymer of glycolide, lactide, caprolactone, trimethylene carbonate, or dioxanone, such as a copolymer of caprolactone and L-lactide, and may include absorbable polyester such as PGA, PLA, PLLA and others like PGLA. In one embodiment, the agent-delivery device may be fabricated out of an absorbable polymer that comprises a therapeutic agent via incorporation of a drug or other therapeutic agent into the base polymer for elution following implantation.

The agent-delivery device may also be fabricated from known biocompatible metals or their alloys such as titanium, stainless steel, cobalt chromium or a combination of multiple types of the materials listed herein.

The agent-delivery device may comprise an amount of a therapeutic agent effective in obtaining a desired local or systemic physiological or pharmacological effect. Suitable therapeutic agents include, but are not limited to, medicaments such as analgesics, anesthetics, antibiotics, antibacterial agents, antifungal agents, anti-inflammatory agents, antimicrobials, antiseptics, bacteriocins, bacteriostats, disinfectants, steroids, antiviral agents, antitumor agents, growth promoting substances, protein antibodies, antioxidants, or mixtures thereof.

Such therapeutic agents for use in combination with the agent-delivery device further include, but are not limited to, acetic acid, aluminum acetate, bacitracin, bacitracin zinc, benzalkonium chloride, benzethonium chloride, betadine, calcium chloroplatinate, certrimide, cloramine T, chlorhexidine phosphanilate, chlorhexidine, chlorhexidine sulfate, chloropenidine, chloroplatinatic acid, ciprofloxacin, clindamycin, clioquinol, cysostaphin, gentamicin sulfate, hydrogen peroxide, iodinated polyvinylidone, iodine, iodophor, minocycline, mupirocin, neomycin, neomycin sulfate, nitrofurazone, non-onynol 9, potassium permanganate, penicillin, polymycin, polymycin B, polymyxin, polymyxin B sulfate, polyvinylpyrrolidone iodine, povidone iodine, 8-hydroxyquinoline, quinolone thioureas, rifampin, rifamycin, copper chloride, copper sulfate, copper peptides, silver acetate, silver benzoate, silver carbonate, silver chloride, silver citrate, silver iodide, silver nitrate, silver oxide, silver sulfate, sodium chloroplatinate, sodium hypochlorite, sphingolipids, tetracycline, zinc oxide, salts of sulfadiazine (such as silver, sodium, and zinc), vitamins such as vitamin E, other agents mentioned above, and mixtures thereof. Preferable bioactive materials are USP approved, more preferably USP monographed.

Additional examples of agents include one or more members selected from the group consisting of anabolic agents, analgesic agents, antiresorptive agents aromatase inhibitors, chondroitin sulphate, COX-2 inhibitors, COX-3 inhibitors, disease modifying anti-rheumatic compounds (DMARDs), glucocorticoids, glucosamine, glycine antagonists, inhibitors of inducible nitric oxide synthetase (iNOS), inhibitors of interleukin-1 converting enzyme, inhibitors of matrix metallo-proteinases (MMPs), inhibitors/antagonists of IL-1, inhibitors/antagonists of RANK-ligand, inhibitors/antagonists of TNF-oc, N-acetylcholine receptor agonists, neurokinin antagonists, neuroleptic agents, NMDA receptor antagonists, non-steroidal anti-inflammatory agents (NSAIDs), opioids, pallitative agents, PAR2 receptor antagonists, selective estrogen receptor modulators (SERMs), vanilloid receptor antagonists, anti-infectives, anti-inflammatories, antioxidants, chlorhexidine, silver sulfadiazine, glycosaminoglycans, natural and truncated forms of parathyroid hormone (PTH), aminated natural and truncated forms of parathyroid hormone (PTH), parathyroid hormone related peptide (PTHrP), anabolic Vitamin D analogs, low-density lipoprotein receptor-related protein 5, non-genomic estrogen-like signaling activator, bone morphogenic protein (BMP), insulin-like growth factor (IGF), fibroblast growth factor (FGF), sclerostin, leptin, a prostaglandin, statin, growth hormone, growth hormone releasing factor (GHRF), hepatocyte growth factor (HGF), calcitonin gene related peptide (CGRP), transforming growth factor (TGF)-.beta.1, human calcitonin, non-human calcitonin, calcitonin gene related peptide (CGRP), hormone replacement therapy (HRT) agents, selective estrogen receptor modulators, bisphosphonates, divalent sources of strontium, fusidic acid, cathepsin-K inhibitors, and antibiotics such as rifampicin, gentamicin, vancomycin and others broadly including bacteriocidal antibiotics such as those which target the bacterial cell wall (penicillins, cephalosporins), or cell membrane (polymixins), or interfere with essential bacterial enzymes (quinolones, sulfonamides), bacteriostatic antibiotics which target protein synthesis, such as the aminoglycosides, macrolides and tetracyclines, and newer antibiotics including the three classes: cyclic lipopeptides (daptomycin), glycylcyclines (tigecycline), and oxazolidinones (linezolid).

Particularly preferred therapeutic agents for use in combination with fracture fixation devices include agents capable of modifying bone healing and remodeling, for example, one or more of calcium salts, strontium salts, vitamin D2 or D3, alphacalcidol, calcitriol or dihydrotachysterol, parathyroid hormone (PTH), bisphosphonates, calcitonin, selective estrogen receptor modulators (SERMs), tissue-specific synthetic steroid analog (a selective tissue estrogenic activity regulator-STEAR), bone morphogenic protein (BMP), glucosamine sulphate and/or other glucosamine containing substances, and/or glucagon like peptide 2 (GLP-2). Other growth factors for bone formation besides BMP may include members of the insulin-like growth factor family, platelet-derived growth factor family, fibroblast growth factor family, transforming growth factor family Proteins important to bone formation include collagens, matrix proteogylcans, osteopontin, alkaline phosphatase, and cell surface attachment molecules like integrins and cadherins. Local regulators of bone include interleukins, prostoglandins, and epidermal growth factor.

Other agents of interest may include, but are not limited to, steroids, pain medication and human monoclonal antibodies such as anti-Tumor Necrosis Factor alpha 1.

One or more therapeutic agents may be located within, or optionally on, the agent-delivery device. For example, the therapeutic agents can be dispersed in the agent-delivery device, such as by being absorbed, or adsorbed, contained, chemically bound, physically bound, or combinations thereof to the agent-delivery device. In addition, the therapeutic agents can be either immobilized on the agent-delivery device, for example so that the agent has a desired effect but is not detached from the material of the device during use, or the agent can be attached to the agent-delivery device in a manner such that the agent becomes detached during use. It is understood that any surface or combination of surfaces, of the agent-delivery device herein described may be the site of the therapeutic agent. Further, the agent-delivery device may be manufactured to provide an immediate, continuous or sustained drug delivery profile.

In another embodiment, the agent-delivery device may comprise at least one portion permeable to the passage of therapeutic agent, allowing diffusion of the agent out of the agent-delivery device. One or more portions of the agent-delivery device may further comprise an impermeable section at least partially surrounding the permeable portion. For example, the agent-delivery device may be formed of an impermeable outer layer at least partially surrounding a permeable portion. A section of the impermeable outer layer may be configured for removal for controlled diffusion of the agent. Alternatively, the impermeable section may contain pores, or openings, of a size capable of providing a targeted agent-delivery profile.

In another embodiment, the agent-delivery device may contain a removable cover or lid to expose the agent or the permeable sections of the agent-delivery device. The cover may be configured to be removed immediately before or after adapting the agent-delivery device to the bone fixation device, for example, just prior to surgically implanting the fixation device.

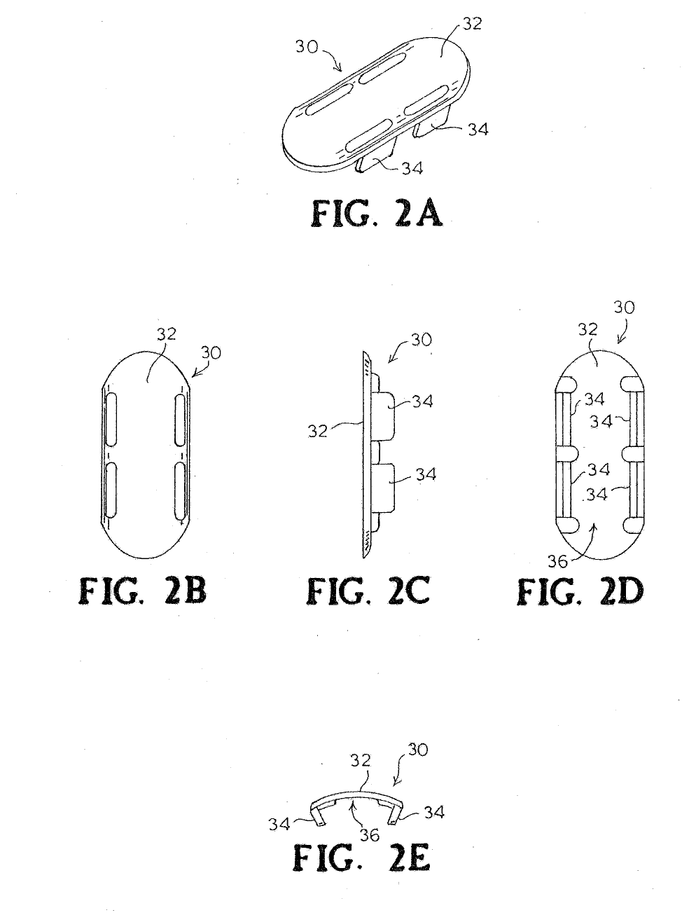

In one aspect, the agent-delivery device may be configured to adapt to a medical implant, such as a fracture fixation plate, by sliding onto the medical implant. One embodiment of a “slide-on” device is shown in FIGS. 2A-2E and generally designated at 30. The agent-delivery device 30 is an elongated member having a substantially oval profile and comprises a substantially major base portion 32 having a longitudinal axis. The base portion 32 spans between generally planar side walls 34, two of which depend from the edges of each side of the base portion 32. The base portion and the side walls define an open longitudinal channel 36. As shown in the FIGs., the side walls 34 are angled inwardly relative to the base portion 32.

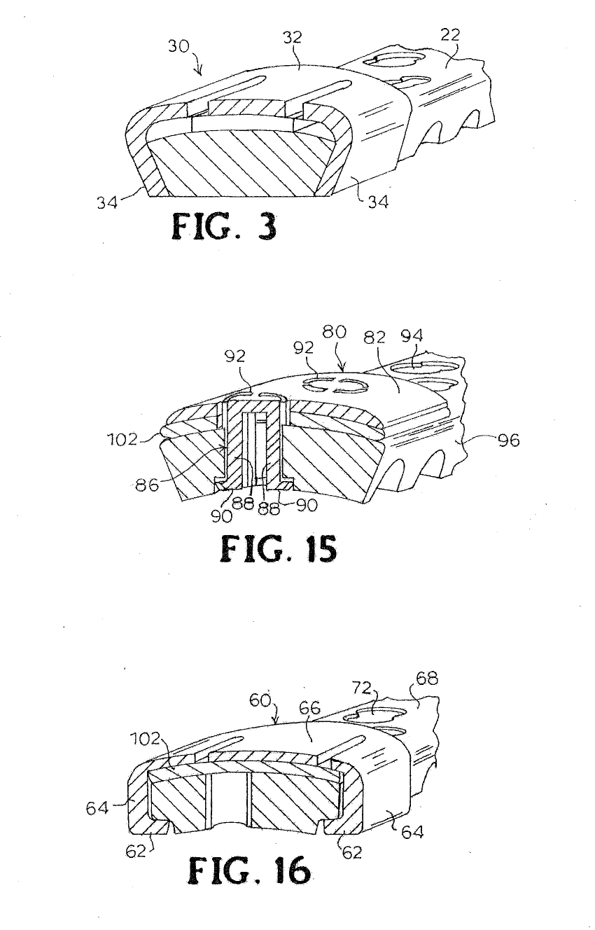

Referring to FIG. 3, the agent-delivery device 30 having this configuration is adaptable to a fracture fixation plate 38 having an upper surface that is wider than the lower surface (the surface against the bone). In use, the agent-delivery device 30 is adapted to the fracture fixation plate 38 by sliding the device onto the end of the plate. The agent-delivery device 30 may be moved to a desired location along the length of the plate 38 manually or by an instrument such as facilitated by a blunt tamp.

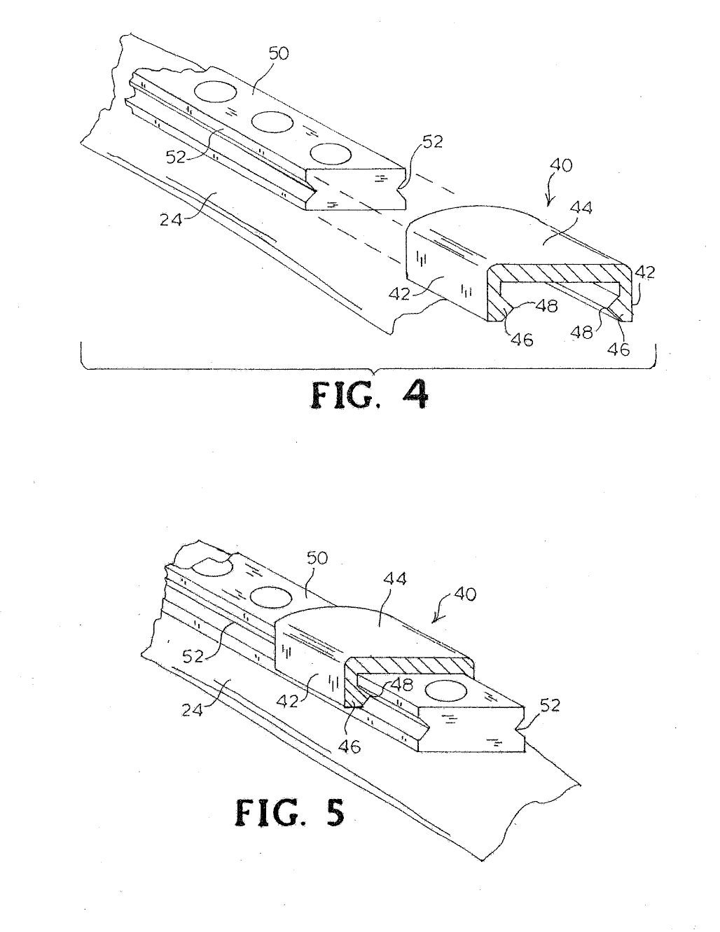

FIG. 4 illustrates another embodiment of a slide-on agent-delivery device and is generally designated at 40. In this embodiment, side walls 42 depend generally perpendicularly along the length of the edges of the base portion 44. In addition, the side walls 42 terminate in flanges 46, which extend inwardly substantially normal to the plane of the side walls 42. The distal ends of the flanges 46 are tapered forming opposed pointed terminal edges 48 which are disposed substantially parallel with respect to the side walls 42. As shown in FIGS. 4 and 5, the sides of the fracture fixation plate 50 define longitudinal grooves 52 corresponding to the pointed edges 48 of the agent-delivery device 40 for slidably receiving the agent-delivery device.

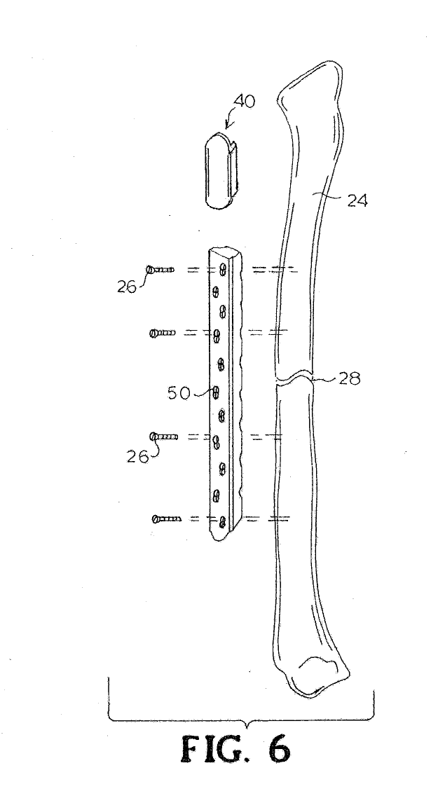

Referring to FIG. 6, in use, the fracture fixation plate 50 is fixed, using surgical screws 26 or other fasteners, to each side of a fracture 28, or otherwise surgically altered site, of a bone 24. The agent-delivery device 40 is adapted by sliding the device onto the end of the fracture fixation plate 50 such that the pointed terminal edges 48 of the flanges 46 are slidably received in the longitudinal grooves 52 in the sides of the plate 50. The agent-delivery device 40 may be advanced along the length of the plate 50 manually or by an instrument such as facilitated by a blunt tamp. The agent-delivery device 40 is positioned so that the agent-delivery device is located proximate to the fracture 28 or the surgical alteration site, as shown in FIG. 1. This is to enable one or more therapeutic agents associated with the device 40 to be delivered to the fracture 28 or site as quickly and efficiently as possible with minimal loss to the system and maximum benefit to the patient. It is understood that the agent-delivery device 40 may be adapted to the fracture fixation plate 50 prior to securing the plate to the bone 24.

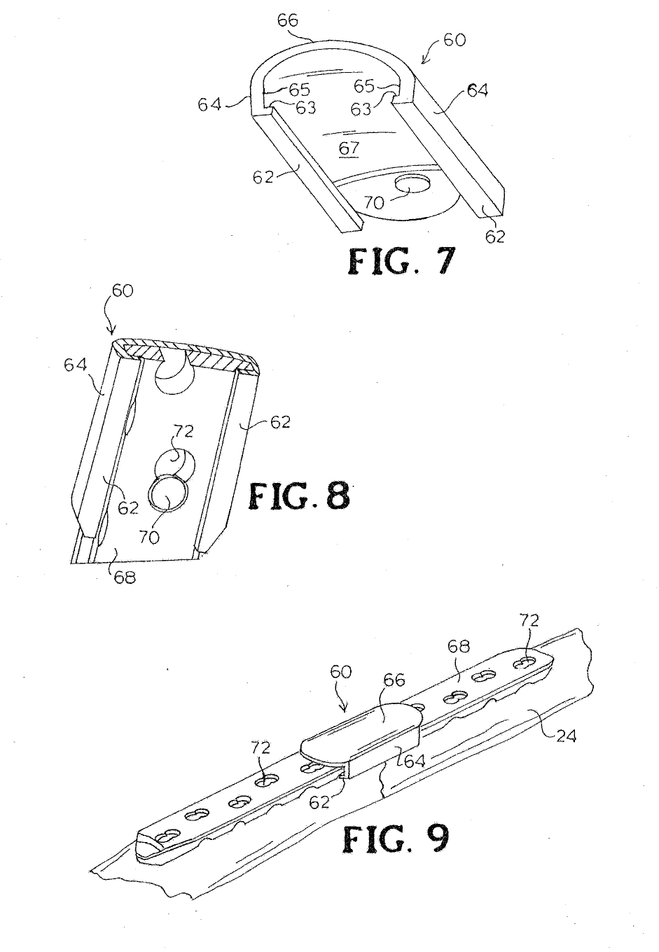

Another embodiment of a slide-on agent-delivery device is shown in FIG. 7 and generally designated at 60. In this embodiment, a continuous inwardly extending flange 62 extends the length of associated side walls 64. The agent-delivery device 60 is sized and shaped such that the distance between the inner surface 65 of the side walls 64 and the distance between the inner surface 67 of the base portion 66 and the upper surface 63 of the flanges 62 is slightly larger than width and thickness, respectively, of the fracture fixation plate 68.

Accordingly, the agent-delivery device 60 is adapted to the fracture fixation plate 68 by sliding the device over the end of the plate, as shown in FIG. 8. In this embodiment, the location of the agent-delivery device 60 relative to the fracture fixation plate 68 may be controlled using a button 70 depending from the inner surface 67 of the base portion 66 of the device (FIG. 7). The button 70 results in one or more areas of increased friction between the agent-delivery device 60 and the plate 68. The button 70 may be sized and shaped to be received within a hole 72 in the plate 68 to prevent relative sliding movement from a desired location. In addition, the agent-delivery device 60 may produce an audible snap as the device is advanced along the fracture fixation plate 68, thereby aiding control of the movement of the agent-delivery device 60 along the plate by providing audible and tactile indicia to the user. Corresponding features could also be formed along the sides of the fracture fixation device 68.

It is understood that in any of the slide-on embodiments described herein, that the pairs of opposed side walls may be sized and shaped to correspond to the sides of the fracture fixation plate so that the agent-delivery may optionally snap into place over the plate. For example, a medical grade polymer material can allow the agent-delivery device to flex sufficiently during installation to accomplish a snap-fit.

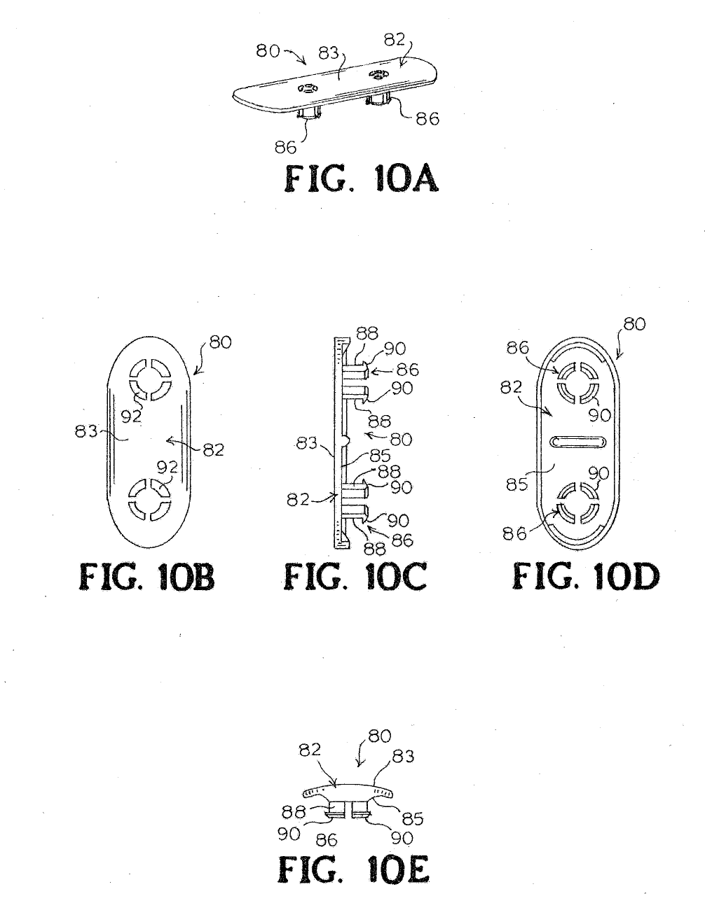

An embodiment of an agent-delivery device configured to “snap-in” at a desired location on a fracture fixation plate is shown in FIG. 10 and generally designated at 80. The agent-delivery device 80 comprises a base portion 82 having a top surface 83 and a bottom surface 85. Two inserts 86 extend at spaced locations from the bottom surface 85 of the base portion 82. Each insert 86 has a cross-section that is generally circular in shape and includes a length measured from the bottom surface 85 of the base portion 82. Each insert 86 comprises four spaced arcuate legs 88. An outwardly extending flange 90 is located at the distal end of each leg 88. A series of arcuate slots 92 define separate rings on the top surface 83 of the base portion 82.

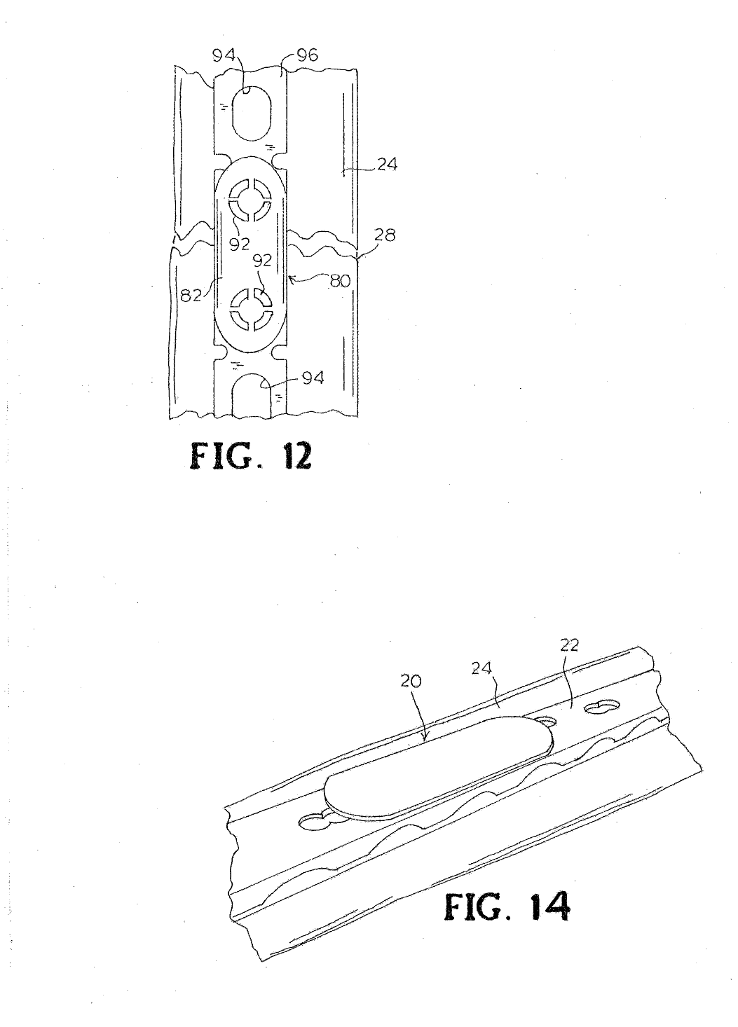

In use, the agent-delivery device 80 is aligned such that the inserts 86 correspond to holes 94 in the fracture fixation plate 96. The agent-delivery device 80 is then pressed in a direction toward the fracture fixation plate 96. The flanges 90 on the legs 88 engage the plate 96 adjacent the holes 94 and, because of the space between each leg, the legs 88 flex inwardly during the downward movement of the device against the fracture fixation plate 96. The inserts 86 thus advance into and through the holes 94 in the fracture fixation plate 96. Once the flanges 90 clear the holes 94 on the other side of the plate 96, the legs 88 of the inserts 86 flex outwardly and the flanges 90 engage the plate. The flanges 90 thus serve to anchor the inserts 86 securely against the fracture fixation plate 96 and prevent any movement of the agent-delivery device 80 relative to the plate. In this manner, the agent-delivery device 80 may be fixed to a portion of the fracture fixation plate 96 on either side of a fracture or surgically altered bone, as shown in FIGS. 11 and 12. It is understood that multiple means of anchorage to the holes in the plate are suitable, including expandable collets that may allow for anchorage to a wide range of hole diameters.

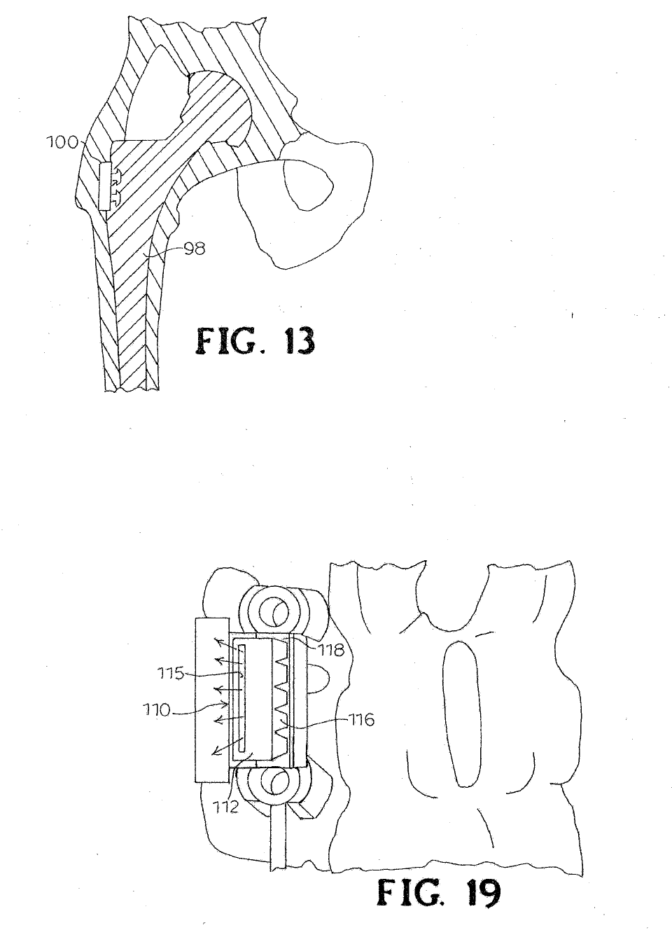

The snap-in agent-delivery device 80 may also be affixed to a long-term implant such as a femoral component 98 of a total hip replacement device, as shown in FIG. 13. In this application, the femoral component 98 of the device may be slightly modified to incorporate an undercut pocket 100 for the snap-in device inserts 86. It is understood that other attachment mechanisms may be used including, but not limited to, a key-way for a slotted insert and the like. In the case of joint arthroplasty, the therapeutic agent may, for example, prevent infection or may accelerate bony ingrowth/ongrowth needed for long-term anchorage in the bone.

In one embodiment, the agent-delivery device may be configured to securely affix to a fracture fixation plate. In one aspect, adhesives are used to secure the agent-delivery device 10 to the fixation device surface (FIG. 14). The adhesives may be absorbable or non-absorbable. Suitable adhesives for use with the agent-delivery device include cyanoacrylates. Examples of cyanoacrylates include, for example, alkyl ester cyanoacrylates, alkyl ether cyanoacrylates or mixtures thereof. For example, suitable adhesives can be prepared by mixing suitable quantities of an alkyl alpha cyanoacrylate such as 2-octyl alpha-cyanoacrylate with one of butyl lactoyl cyanoacrylate (BLCA), butyl glycoloyl cyanoacrylate (BGCA), isopropyl glycoloyl cyanoacrylate (IPGCA), ethyl lactoyl cyanoacrylate (ELCA), and ethyl glycoloyl cyanoacrylate (EGCA). Such mixtures may range from ratios of about 90:10 to about 10:90 by weight, preferably about 75:25 to about 25:75 by weight such as from about 60:40 to about 40:60 by weight.

In one aspect, the agent-delivery device can include a pressure sensitive adhesive on at least a portion of at least one surface, to assist in initial placement of the agent-delivery device on the desired portion of the fixation device. In other aspects, the agent-delivery device includes a pressure sensitive adhesive on at least one side in combination with one or more mechanical securement means, such as herein disclosed. The pressure sensitive adhesive can be covered by a suitable release layer or liner, if desired, to preserve the adhesiveness of the material until time of use. The pressure sensitive adhesive may also include a therapeutic agent.

Referring to FIGS. 15 and 16, a therapeutic agent-eluting sponge 102 may be placed between the base portion of the agent-delivery device and the fracture fixation plate. This feature is applicable to both the snap-in and slide-on embodiments of the agent-delivery device 60, 80. Openings 104 through the base portion in this and other embodiments provide pathways so that the therapeutic agent within the sponge 102 is immediately available to the localized area to deliver the desired therapeutic effect. As described in detail herein, the eluting sponge 102 may include therapeutic agents that are designed to be released from the sponge 102 at a delayed, sustained, or controlled rate into the surrounding area to achieve a particular delivery profile and provide maximum benefit to the patient. Alternatively, a highly viscous gel or a cross-linked gel may be used as the carrier material to fill the cavity and for local delivery of the therapeutic agent.

FIGS. 15 and 16 also show minor modifications to the fracture fixation plate for accommodating the agent-delivery device. Specifically, the fracture fixation plates may be modified to add a small countersink or a slot/chamfer on the underside of the plate to receive the correspondingly configured agent-delivery device.

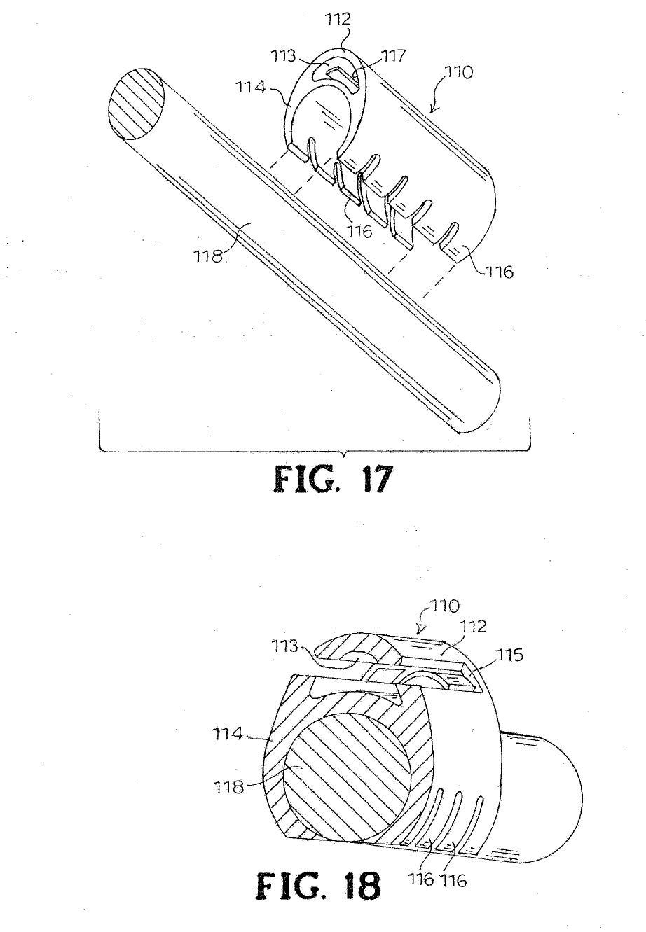

Another embodiment of a snap-on agent-delivery device is shown in FIG. 17 and generally designated at 110. This embodiment of the agent-delivery device 110 comprises a compartment portion 112 and a sleeve 114. The compartment portion 112 defines an open-ended cavity 113 extending along at least a portion of the compartment 112 for accommodating a therapeutic agent-eluting sponge (not shown). The compartment 112 has one or more slots 115 therein that open into and extend along the length of the cavity 113. The sleeve 114 includes a first side and a second side, each side having a series of opposed arcuate fingers 116.

The agent-delivery device 110 is configured to snap-on to a rod 118 used for spinal fixation (FIG. 18). Referring to FIG. 19, in use, the rod 118 is set in place within the spinal column at the site of the spinal instrumentation, for example to accelerate bone healing or in order to treat (or prevent) infection. The user places the agent eluting sponge or drug eluting gel within the cavity 113 of the compartment portion 112. The sleeve 114 is then snapped onto a portion of the length of the rod 118 so that the fingers 116 are gripping the circumference of the rod 118 and hold the sleeve 114 in place such that the compartment portion 112 may be positioned in close proximity to the area in which a bony fusion is desired, such as adjacent vertebrae. Therapeutic agents within the sponge are released via the slots 115 in the compartment 112 or through the open ends 117 of the compartment. In this manner, the agents are released into the area of the fracture site, or fusion construct site, to deliver the desired therapeutic value.

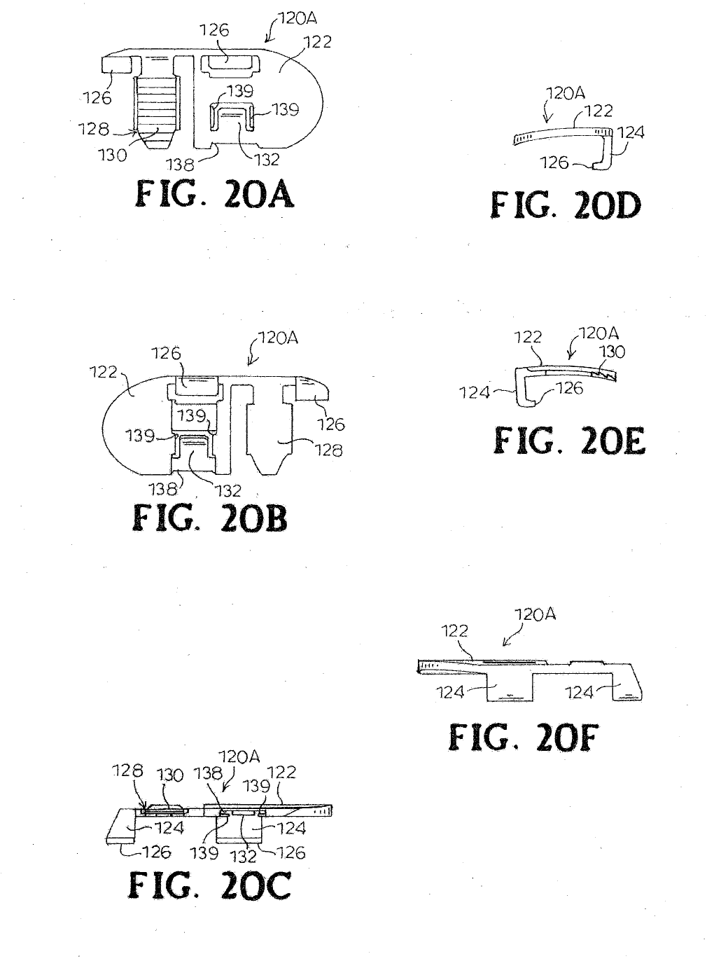

FIGS. 20A to 20F show an embodiment of an adjustable snap-on device, generally designated at 120A, 120B. The body portions 120A, 120B are generally mirror images of one another and include a base portion 122 and depending side walls 124 and inwardly directed flanges 126 at the edges of the side walls 124. Each body portion 120A, 120B also has an inwardly extending tongue 128 in the plane of the base portion 122. Each tongue 128 has transverse ridges 130 along its upper surface. A tab 132 is spaced longitudinally from the tongue 128 on each body portion 120A, 120B and is integral with the base portion 122. Each tab 132 has ridges 134 on its lower surface. The inner side walls 124 of each body portion 120A, 120B define an opening 136 for receiving the tongue 128.

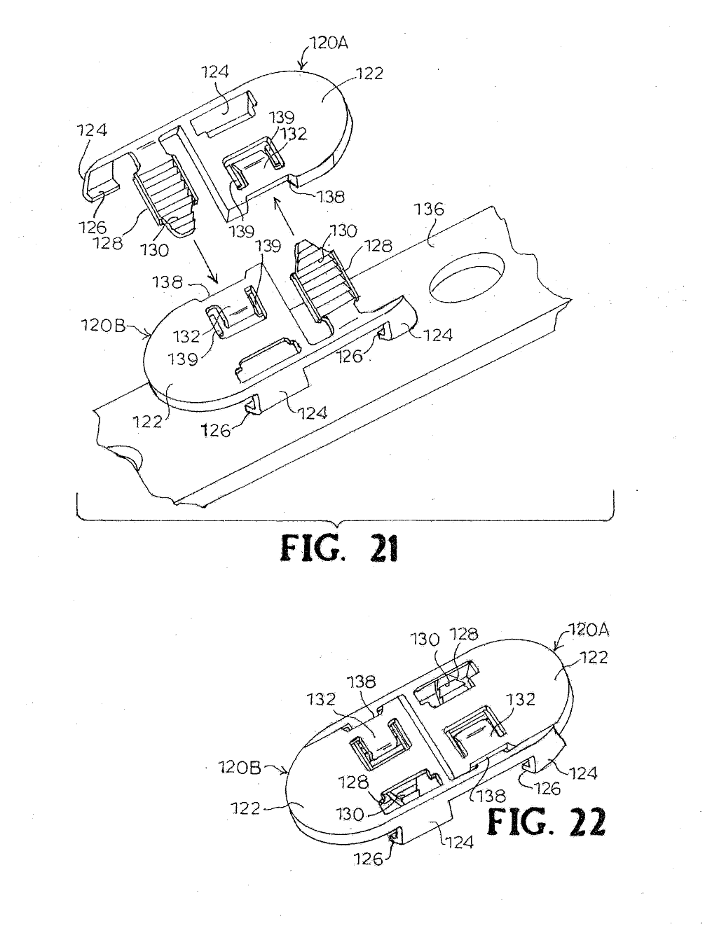

As shown in FIG. 21, the tongues 128 are aligned with the corresponding opening 136 in the body portions 120A, 120B and advanced towards one another in the direction of the arrows. The ridges 130 on the tongues 128 engage the ridges 134 on the tab 134 for securely joining the two body portions 120A, 120B. As seen in FIG. 22, the configuration of the joined body portions 120A, 120B of the agent-delivery device 120 generally now corresponds to the shape of previous embodiments of the device described herein.

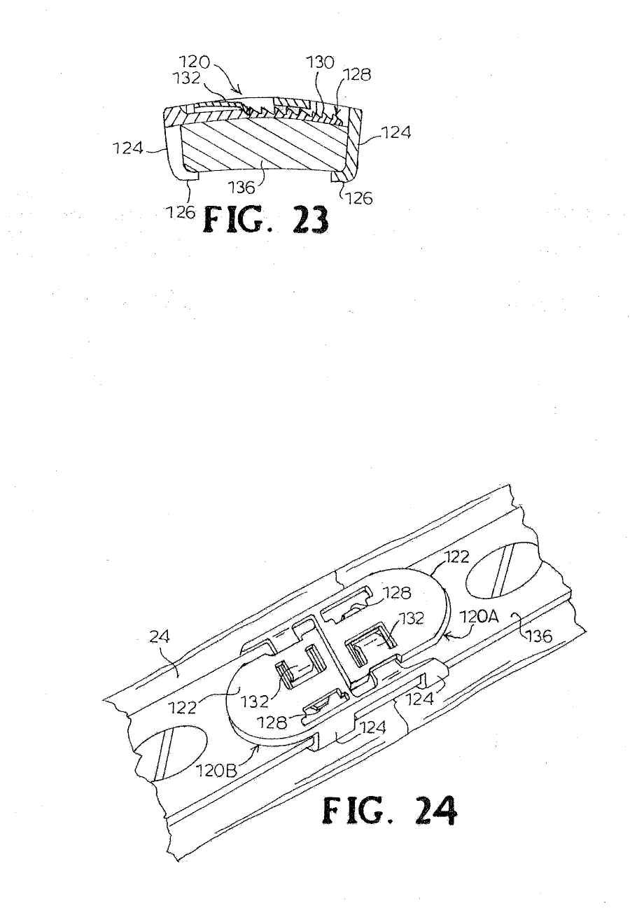

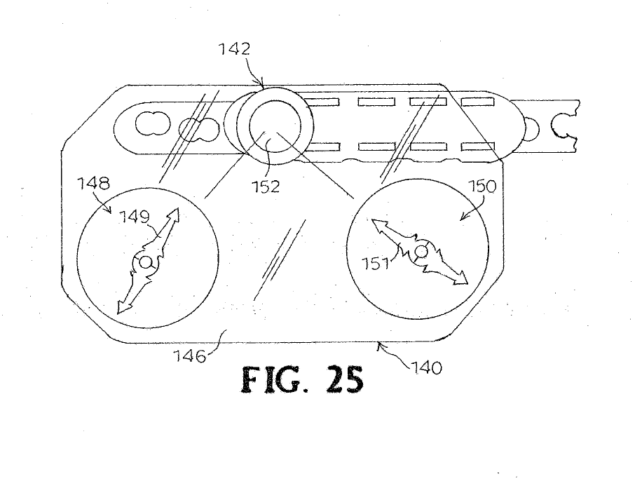

In use, the body portions 120A, 120B of the agent-delivery device 120 can be brought together and secured adjacent the upper surface and sides of a fracture fixation plate 136. The body portions 120A, 120B are advanced towards one another such that the sides of the fracture fixation plate 136 are received in the slots 138 defined by the side walls 124, flanges 126 and lower surface of the base portion 122. As the body portions 120A, 120B are advanced towards one another, the ridges 130 on the tongues 128 engage the ridges 134 on the respective tabs 132 to form a secure fit on the fraction fixation plate 136. This arrangement can be seen in FIGS. 23 and 24.

It is understood that the size of the embodiments of agent-delivery device depicted herein re merely exemplary and that the size may vary as suitable for a particular indication. For example, the agent-delivery device may be sized to substantially cover a fracture fixation plate in order to ensure delivery of therapeutic agent locally to the entire area around the plate. Thus, the applicants do not intend to be limited to the relative sizes of the agent-delivery devices shown herein. Similarly, the same goal can be accomplished by using a plurality of agent-delivery devices along the length of the fracture fixation plate, or other medical implant, as desired.

In one embodiment, an agent-delivery device may define a reservoir adapted to contain a therapeutic agent effective in obtaining a desired local or systemic physiological or pharmacological effect. The reservoir may be integral with or separable from the agent-delivery device. The reservoir, or a portion of the reservoir, may comprise a permeable material which is contained in a substantially impermeable portion of the device. For example, the reservoir may comprise an impermeable outer layer around a permeable material comprising the agent, allowing diffusion of the agent out of the reservoir. The impermeable portion of the reservoir may optionally contain pores of a size capable of providing a targeted delivery profile. The reservoir may comprise a carrier, such as a sponge or gel material, capable of absorbing or adsorbing or otherwise containing the therapeutic agent. A removable cover or lid may be adapted to be removed as desired to expose the carrier or a permeable portion of the reservoir. For example, a section of the impermeable outer layer of the reservoir may be configured for removal. The cover may be configured to be removed to introduce one or more agents to the reservoir, or immediately before or after adapting the agent-delivery device to the fixation device, for example, just prior to surgically implanting the fixation device.

An integral, resealable valve may be provided to allow the reservoir to be filled by a physician during a postoperative, outpatient procedure without surgical intervention. Filling of the reservoir may be accomplished by percutaneous injection through the valve into the reservoir. An external valve-location means may be provided to accurately locate the position of the valve among the surrounding tissue.

An embodiment of a resealable valve and valve locator combination is shown in FIG. 25, comprising an agent-delivery device including a reservoir and a locator, generally shown at 140. The agent-delivery device comprises a resealable valve 142 which is designed to operate with the external locator 140, allowing a surgeon to determine the position of the valve for post-operative injections to fill the reservoir with a desired therapeutic agent. The valve 142 is provided with indicia in the form of magnetically-responsive elements 144, such as magnets, although other metallic elements could be used provided they are magnetically-responsive, as well as any other means to signify the position of the valve which are capable of being determined by external locator devices.

The locator comprises a base 146, including a plurality of sensors 148, 150, each of which may comprise a magnetic compass needle. Each needle is allowed to freely orientate with either the north or south magnetic pole within a closed recess in the base 146. The sensors 148, 150 are spaced from one another such that when the locator 140 is maneuvered into position over the valve 142 the pair of north or south indicating needles 148, 150 orientate with one another and define a third point 152, shown by the target opening which indicates a true position of the valve 142. FIG. 25 shows the needles 148, 150 pointing toward the target 152 to signify the true location of the underlying valve 142. Thus, a physician (or nurse) is able to precisely locate the injection valve.

It is understood that the injection valve may be situated at a location remote from the medical implant, and the valve coupled with a fill tube feeding into the reservoir, whereby agent injected into the valve flows through the fill-tube into the reservoir.

A suitable arrangement of this type, including a resealable valve and locator means, is described in U.S. Pat. No. 5,146,933, the contents of which are hereby incorporated by reference in their entirety.

As described herein, the agent-delivery device allows for a highly localized delivery of one or more therapeutic agents. Without being bound by any particular theory, it is believed that the therapeutic agent associated with the device is released into the body locally proximate to a fracture site. The mechanism of action in a fracture repair is generally the diffusion of the therapeutic agent inward, toward the separated bony regions and the central intramedullary canal. This is the site at which primary or secondary healing of the separated bony surfaces will occur during the fracture repair and bone fusion process. The diffusion process may be facilitated by the holes in the fracture fixation device at the fracture site, for example, those which are not occupied by anchoring screws. In addition to diffusion of the agent toward the fracture healing site, diffusion may also occur outward along the outer periosteal surface of the bone and the outer surface of the fracture callus that forms at the site of fracture repair. Thus, the therapeutic agent is delivered with maximum efficiency to the needed area to enhance bone growth, decrease swelling, minimize pain, fight infection, or any number of other therapeutic achievements.

A plurality of therapeutic agents may be utilized depending on the particular situation or as determined by a healthcare provider. The agent-delivery device may be configured to provide diffusion from specific portions, or surfaces, thereof of one or more therapeutic agents in proximity to one or more specific tissues. For example, an antibiotic may be allowed to diffuse outward into a region around the plate in order to prevent infection at the site of the fracture, while a growth factor may diffuse inwards to accelerate the recruitment of bone precursor cells needed for bone formation and fracture incorporation.

The agent-delivery devices and methods described herein have many advantages, including allowing the surgeon to achieve intra-operative antibiotic resistance, such as in open fractures or other environments of high risk for infection. Alternatively, the agent-delivery device may be easily affixed to an implant at a later time, such as during a debridement and exploration of an infected implant. The local or sustained delivery via the described technology is cost effective. For example, when a fixation device with an agent-delivery device comprising a reservoir is employed, the ability to easily and conveniently affix or replenish the agent-delivery device or the reservoir will likely not delay the operative procedure or increase the operating room time and expense. Cost savings may be achieved via reduced post-operative hospitalization time, reduced likelihood of a revision surgery, for either infection or pseudoarthrosis, and more rapid patient recovery and return to work. Drugs or protein therapies may be conserved by locally delivering a targeted dosage of the therapeutic agent desired. More rapid healing should result in reduced narcotic usage by the patient, and the fixation device with an agent-delivery device may also allow for local delivery of pain-relieving substances into the local environment as opposed to high dosages of systemic narcotics or NSAIDs.

The agent-delivery device is easily adapted to or incorporated into an implant system already in clinical use. Commercially available fracture fixation plates are suitable for use with the agent-delivery device. The device is able to be adapted to affix to a wide range of off-the-shelf medical devices without a requirement to significantly modify the implant for receipt of the local delivery agent. At the same time, agent-delivery devices described herein may be fabricated that adapt to atypical or custom fixation plates with little or no modification to the plate required. The agent-delivery device may be configured to securely adapt to the geometry of the fixation device.

Because the agent-delivery device is entirely separate from the, usually, metallic fixation implant. The two components of a delivery system may be separately constructed, packaged, stored and processed. This allows for separate sterilization of the two systems, should each require differing means of packaging and sterilization. For example, metallic devices are robust and can be sterilized using high doses of radiation or heat and steam. Polymeric materials and therapeutic agents are more fragile and may require low doses of ionizing radiation or gas for sterilization. A therapeutic drug may be processed aseptically rather than undergo a terminal sterilization step. The therapeutic drug, for example a protein growth factor, may be added to the agent-delivery device either in advance of the surgery or at the time of surgery. This will allow the healthcare practitioner to select the agent of interest and dosing required that will be tailored to the patient and the implant environment.

Surgeons may utilize the implants in a standard fashion, including rather vigorous handling of the devices during templating, sizing and implant insertion. In some cases, the implant may be shaped or bent to conform to the body at the time of surgery. The agent-delivery device may be fixed to the implant at the time of surgery or at a later time, such as in the case of revision for infection or non-fusion. In some embodiments, a reservoir containing the therapeutic agent is filled at the time of surgery (or at later follow-up), allowing the surgeon great intra-operative flexibility to select the required antibiotic, growth factor or other agent at the time of surgery.

Although the agent-delivery device has been shown and described in considerable detail with respect to only a few exemplary embodiments thereof, it should be understood by those skilled in the art that we do not intend to limit the invention to the embodiments since various modifications, omissions and additions may be made to the disclosed embodiments without materially departing from the novel teachings and advantages of the invention, particularly in light of the foregoing teachings. For example, the agent-delivery devices described herein are generally applicable to other implant devices in addition to internal fracture fixation devices. Accordingly, we intend to cover all such modifications, omission, additions and equivalents as may be included within the spirit and scope of the invention as defined by the following claims. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

We claim:

1. An device for use with a medical implant for delivering an agent to a designated site of action in a body of a patient, the agent-delivery device comprising:
a body member adapted to be secured to the medical implant; and
an agent-delivery component associated with the body member, the agent-delivery component including a therapeutic agent for treating the body of the patient,
wherein the agent-delivery component is configured to release the therapeutic agent after implantation in the body of the patient.

2. An agent-delivery device as recited in claim 1, wherein the body member comprises
a base portion terminating in longitudinal edges; and
legs extending from the longitudinal edges of the base portion and terminating in longitudinal edges, the legs extending from the base portion at an acute angle such that the base portion and the legs define an open longitudinal channel narrower at the edges of the legs than at the base portion,
wherein the longitudinal channel is adapted to slidably receive the medical implant.

3. An agent-delivery device as recited in claim 1, wherein the body member comprises
a base portion terminating in longitudinal edges;
legs extending from the longitudinal edges of the base portion and terminating in longitudinal edges, the base portion and the legs defining an open longitudinal channel; and
a flange integral with the longitudinal edges of each leg, the flanges extending inwardly such that the base portion, legs, and flanges define opposed longitudinal slots,
wherein the longitudinal channel and slots are adapted to receive the medical implant.

4. An agent-delivery device as recited in claim 1, wherein the body member comprises a bio-absorbable polymer.

5. An agent-delivery device as recited in claim 1, wherein the body member comprises a non-bio-absorbable polymer.

6. An agent-delivery device as recited in claim 1, wherein the agent-delivery comprises a carrier for the therapeutic agent.

7. An agent-delivery device as recited in claim 6, wherein the carrier comprises a sponge.

8. An agent-delivery device as recited in claim 1, wherein the agent-delivery component comprises at least a portion of the surface of the body member having the therapeutic agent impregnated thereon or adsorbed thereon or otherwise contained therein, and wherein the agent desorbs, elutes, or is otherwise delivered from the body member to a site of action.

9. An agent-delivery device as recited in claim 1, further comprising an insert having an axis defined in a direction of insertion, the insert comprising:
a plurality of legs depending from the inner surface of the body member and along the insert axis, the outer dimensions of the legs having a profile adapted to be received in a hole in the medical implant, the length of the legs adapted to be longer than the thickness of the area of the medical implant defining the hole; and
outwardly extending flanges at a distal end of the legs, the outer dimensions of the flanges having a wider profile than the legs relative to the insertion axis,
wherein the insert is adapted to be received in the hole in the medical implant, the flanges adapted for contacting the implant adjacent the hole during insertion for flexing the legs resiliently inwardly for reducing the outer dimensions of the flanges such that the flanges pass through the hole and the flanges contact the opposite side of the medical implant adjacent the hole for a snap-fit securing the agent-delivery device relative to the medical implant.

10. An agent-delivery device as recited in claim 1, further comprising:
opposed arcuate walls depending from a surface of the body member, the walls defining a partial tube and a longitudinal slot extending between the length of the terminal edges of the walls,
wherein the walls are adapted to receive a cylindrical medical implant, the walls flexing resiliently outwardly when pressed against the medical implant for enlarging the slot and receiving the medical implant between the walls such that the agent-delivery device is secured relative to the medical implant.

11. An agent-delivery device as recited in claim 1, wherein the body member comprises
a first base portion and a second base portion, each base portion terminating in longitudinal edges defining a transverse slot in one edge, and including
at least one leg extending from the edge of each base portion opposite the slot and terminating in a longitudinal edge,
a flange integral with the longitudinal edge of the at least one leg, the flange extending inwardly such that each base portion, the at least one leg, and the flange define a longitudinal slot for receiving a medical implant,
a tongue integral with each base portion, and
means operatively associated with each slot for securing the tongue at a plurality of relative positions of the base portions,
wherein the tongues are configured to be received in a respective slot for securing together the first base portion and the second base portion such that the first and second base portions and the legs define an open longitudinal channel adaptable to the width of the medical implant.

13. An agent-delivery device as recited in claim 1, further comprising an agent-delivery system including at least one reservoir and having a valve in fluid communication with an orifice opening into the reservoir.

14. An agent-delivery device as recited in claim 13, further comprising a magnetic element associated with the valve, wherein when the device has been placed within the body of a patient, the location of the valve can be located from outside the body of the patient using a triangulation technique for introducing the agent to the reservoir via the valve by a tubular member.

15. An agent-delivery device as recited in claim 13, wherein the tubular member is selected from a cannula, a rod, a shaft, and a needle.

16. A system for use as a medical implant, the medical implant system comprising:
a medical implant;
a device for delivering an agent to a designated site of action in a body of a patient, the agent-delivery device comprising:
a body member configured to be secured to the medical implant, and
an agent-delivery component associated with the body member, the agent-delivery component including a therapeutic agent for treating the body of the patient,
wherein the agent-delivery component is configured to release the therapeutic agent after implantation in the body of the patient.

17. A medical implant system as recited in claim 16, wherein the medical implant comprises a bone fixation element adapted to be secured to a bone portion in the body of a patient.

18. A medical implant system as recited in claim 16, wherein the agent-delivery device is attached to the medical implant with an adhesive.

19. A medical implant system as recited in claim 16, wherein the body member comprises
a base portion terminating in longitudinal edges; and
legs extending from the longitudinal edges of the base portion and terminating in longitudinal edges, the legs extending from the base portion at an acute angle such that the base portion and the legs define an open longitudinal channel narrower at the edges of the legs than at the base portion,
wherein the longitudinal channel is configured to slidably receive at least a portion of the medical implant.

20. A medical implant system as recited in claim 16, wherein the body member comprises
a base portion terminating in longitudinal edges;
legs extending from the longitudinal edges of the base portion and terminating in longitudinal edges, the base portion and the legs defining an open longitudinal channel; and
a flange integral with the longitudinal edges of each leg, the flanges extending inwardly such that the base portion, legs, and flanges define opposed longitudinal slots,
wherein the longitudinal channel and slots are configured to slidably receive the medical implant.

21. A medical implant system as recited in claim 20, wherein the flanges terminate in pointed longitudinal edges, and wherein the medical implant has V-shaped grooves in exterior surfaces for receiving the edges of the flanges.

22. A medical implant system as recited in claim 20, wherein the body member further comprises a projection extending into the channel, and the medical implant defines an opening for receiving the projection for securing the agent-delivery device relative to the implant.

23. A medical implant system as recited in claim 16, wherein the body member comprises a bio-absorbable polymer.

24. A medical implant system as recited in claim 16, wherein the body member comprises a non-bio-absorbable polymer.

25. A medical implant system as recited in claim 16, wherein the agent-delivery comprises a carrier for the therapeutic agent.

26. A medical implant system as recited in claim 25, wherein the carrier comprises a sponge.

27. A medical implant system as recited in claim 16, wherein the agent-delivery component comprises at least a portion of the surface of the body member having the therapeutic agent impregnated thereon or adsorbed thereon or otherwise contained therein, and wherein the agent desorbs, elutes, or is otherwise delivered from the body member to a site of action.

28. A medical implant system as recited in claim 16, wherein the medical implant has at least one hole, and further comprising an insert having an axis defined in a direction of insertion, the insert comprising:
a plurality of legs depending from the inner surface of the body member and along the insert axis, the outer dimensions of the legs having a profile adapted to be received in a hole in the medical implant, the length of the legs adapted to be longer than the thickness of the area of the medical implant defining the hole; and
outwardly extending flanges at a distal end of the legs, the outer dimensions of the flanges having a wider profile than the legs relative to the insertion axis,
wherein the insert is configured to be received in the at least one hole in the medical implant, the flanges contacting the implant adjacent the hole during insertion for flexing the legs resiliently inwardly for reducing the outer dimensions of the flanges such that the flanges pass through the hole and the flanges contact the opposite side of the medical implant adjacent the hole for a snap-fit securing the agent-delivery device relative to the medical implant.

29. A medical implant system as recited in claim 28, wherein the medical implant comprises a bone fixation member defining a plurality of holes spaced longitudinally along the member and adapted for receiving fasteners for securing the member to bone.

30. A medical implant system as recited in claim 16, wherein the medical implant comprises a rod portion, and the body member further comprises opposed arcuate walls depending from a surface of the body member, the walls defining a partial tube and a longitudinal slot extending between the length of the terminal edges of the walls, wherein the walls are adapted to receive the rod, the walls flexing resiliently outwardly when pressed against the rod for enlarging the slot and receiving the rod between the walls such that the agent-delivery device is secured relative to the medical implant.

31. A medical implant system as recited in claim 16, wherein the body member comprises
a first base portion and a second base portion, each base portion terminating in longitudinal edges defining a transverse slot in one edge, and including
at least one leg extending from the edge of each base portion opposite the slot and terminating in a longitudinal edge,
a flange integral with the longitudinal edge of the at least one leg, the flange extending inwardly such that each base portion, the at least one leg, and the flange define a longitudinal slot for receiving the medical implant,
a tongue integral with each base portion, and
means operatively associated with each slot for securing the tongue at a plurality of relative positions of the base portions,
wherein the tongues are configured to be received in a respective slot for securing together the first base portion and the second base portion such that the first and second base portions and the legs define an open longitudinal channel adjustable to the width of the medical implant.

32. A medical implant system as recited in claim 16, further comprising an agent-delivery system including at least one reservoir for accommodating a therapeutic agent, and a valve in fluid communication with an orifice opening into the reservoir.

33. A medical implant system as recited in claim 32, further comprising a magnetic element associated with the valve, wherein when the device has been placed within the body of a patient, the location of the valve can be located from outside the body of the patient using a triangulation technique for introducing the agent to the reservoir via the valve by a tubular member.

34. A medical implant system as recited in claim 33, wherein the tubular member is selected from a cannula, a rod, a shaft, and a needle.

35. A method of delivering a therapeutic agent to a site within the body of a patient adjacent to a medical implant, the method comprising the steps of:
providing a medical implant;
providing a therapeutic agent to be delivered to a site of action within the body of a patient;
providing an agent-delivery device;
operatively associating the therapeutic agent with the agent-delivery device such that the therapeutic agent is arranged to delivery a therapeutically effective amount of the agent to the site;
securing the agent-delivery device to the medical implant; and
surgically implanting the medical implant.

36. A method of delivering a therapeutic agent as recited in claim 35, wherein the step of providing an agent-delivery device comprises providing an agent-delivery device including a body member comprising a base portion terminating in longitudinal edges, and legs extending from the longitudinal edges of the base portion and terminating in longitudinal edges, the legs extending from the base portion at an acute angle such that the base portion and the legs define an open longitudinal channel narrower at the edges of the legs than at the base portion; and
wherein the securing step comprises sliding the agent-delivery device onto the medical implant such that the longitudinal channel receives at least a portion of the medical implant.

37. A method of delivering a therapeutic agent as recited in claim 35, wherein the step of providing an agent-delivery device comprises providing an agent-delivery device including a body member comprising a base portion terminating in longitudinal edges, legs extending from the longitudinal edges of the base portion and terminating in longitudinal edges, the base portion and the legs defining an open longitudinal channel, and a flange integral with the longitudinal edges of each leg, the flanges extending inwardly such that the base portion, legs, and flanges define opposed longitudinal slots; and
wherein the securing step comprises sliding the agent-delivery device onto the medical implant such that the longitudinal channel and slots slidably receive at least a portion of the medical implant.

38. A method of delivering a therapeutic agent as recited in claim 35, wherein the step of providing an agent-delivery device comprises providing an agent-delivery device including an insert having an axis defined in a direction of insertion, the insert comprising a plurality of legs depending from the inner surface of the body member and along the insert axis, the outer dimensions of the legs having a profile adapted to be received in a hole in the medical implant, the length of the legs adapted to be longer than the thickness of the area of the medical implant defining the hole, and outwardly extending flanges at a distal end of the legs, the outer dimensions of the flanges having a wider profile than the legs relative to the insertion axis; and
wherein the securing step comprises directing the insert into a hole in the medical implant, the flanges contacting the implant adjacent the hole during insertion for flexing the legs resiliently inwardly for reducing the outer dimensions of the flanges such that the flanges pass through the hole and the flanges contact the opposite side of the medical implant adjacent the hole for a snap-fit.

39. A method of delivering a therapeutic agent as recited in claim 35, wherein the securing step comprises attaching the agent-delivery device to the medical implant with an adhesive.

40. A method of delivering a therapeutic agent as recited in claim 35, wherein the step of providing a medical implant comprises providing a bone fixation member defining a plurality of holes spaced along the member and adapted for receiving fasteners for securing the member to bone.

41. A method of delivering a therapeutic agent as recited in claim 35, further comprising the step of providing an agent-delivery system including at least one reservoir for accommodating a therapeutic agent, and a valve in fluid communication with an orifice opening into the reservoir; and
delivering therapeutic agent to the reservoir post-implantation.

42. A method of delivering a therapeutic agent as recited in claim 35, further comprising the step of providing a magnetic element associated with the valve, and
locating the valve from outside the body of the patient using a triangulation technique for introducing the agent to the reservoir via the valve by a tubular member.
